(19) United States
(12) Patent Application Publication
(10) Pub. No.: US 2017/0296059 A1
(43) Pub. Date: Oct. 19, 2017

(54) EIT 3D scanning Clothing

(71) Applicant: Timothy John Anderson, Minneapolis, MN (US)

(72) Inventor: Timothy John Anderson, Minneapolis, MN (US)

(21) Appl. No.: 15/170,415

(22) Filed: Jun. 1, 2016

Related U.S. Application Data
(60) Provisional application No. 62/230,323, filed on Jun. 2, 2015.

(51) Int. Cl. A61B 5/00; G06F 19/00

(52) U.S. Cl. CPC A61B 5/0033; G06F 19/3431; G06F 19/3406; G06F 19/3418

(57) ABSTRACT

The present invention relates to Electrical Impedance Tomography (hereinafter “EIT”) wearable technology and Telemedicine to provide cloths that can scan and image the human body safely and affordable detecting and monitoring cancer, pneumonia and a patient's medical condition in real time anywhere in the world with an Internet connection.

### DETAILED DESCRIPTION OF THE PREFERRED EMBODIMENTS

A person puts on clothing or wearable technology that has a network of Electrical Impedance Tomography sensors integrated into it the sensors send electrical current through the body and observes the resistance it incurs while passing through the body the network connect to a smart device through Bluetooth or a cord the smart device opens an app connects to the cloud, computers in the cloud process the data and send an image back of the internal body of the person the electrical impedance cancerous tumor's, fluid on the Lungs and other health problems are highlighted and tracked along with a person body fat percentage this can be monitored on a daily basis.

### FIELD OF THE INVENTION

Invention relates generally to Telemedicine, Electrical Impedance Tomography and Wearable Technology.

### BACKGROUND

Electrical impedance tomography has been used for years in scales to tell a person body mass index and in hospitals and clinics to image the internal human body primarily to monitor lung function our invention improves upon this by integrating EIT with wearable technology and telemedicine to make self body scanning system for caner and lung function affordable and save for anybody with an Internet connection.

Cancer detection today generally requires that a patient show signs of sickness to the point that a doctor will request an x-ray or an MRI the goal of our technology is to use electrical impedance tomography integrated into clothing or other wearable technology to let people do self cancer screening of their own body whenever and wherever they want in an affordably and safely manner this will save lives and reduced cost.

Cancer patients in remission today have to wait up to six months to a year to get new x-rays or MRI's to see if the cancer has come back our technology will allow them to scan them selves every day to see if there are cancer free or monitor the progress of the treatment and thank to the telemedicine it can be done from the comfort of their own home.

Pneumonia is one of the leading killers of senior citizens and children around the world our invention will allow early detection and monitoring of pneumonia this will save lives reduce suffering and drive down healthcare costs around the planet.

### SUMMARY OF THE INVENTION

A network of electrical impedance tomography sensors integrated in to clothing that use a smart device to connect to the cloud to scan the human body for cancer, pneumonia and other health issues and create a multi dimensional image that a person can see of their own body and telemedicine would allow doctor and healthcare provider to monitor patients anywhere they are in the world from home or from a hospital bed.

### EMBODIMENT OF THE INVENTION

A network of Electrical Impedance Tomography sensors integrated into wearable tech-knowledge or clothing using electrical current to image the internal human body this scanning system could detect hard cancerous tumors, pneumonia on the Lungs, body fat percentage, identify the location of excess fat in the body and other health issues inside of the body the sensors are connected to a smart device like smart phone or other portable computer that connects to the cloud the processing power of the cloud is used to process the data and then the images sent back to the phone or smart device and to the person Doc if they choose.

### BREIF DESCRIPTION OF THE DRAWING

FIG. 1 shows a shirt with a network of sensor integrated into its connected to a smart phone that's connected to the cloud

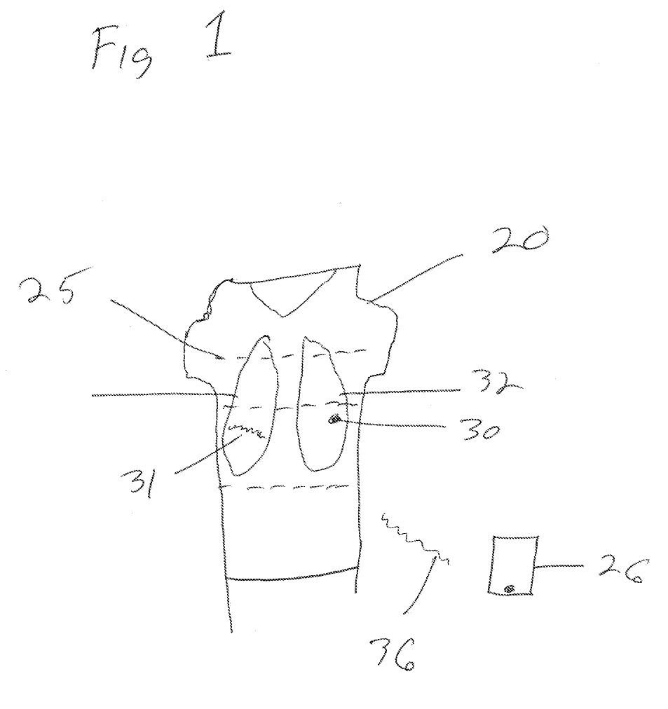

FIG. 2 shows a blanket connected to a smart device connected to the cloud

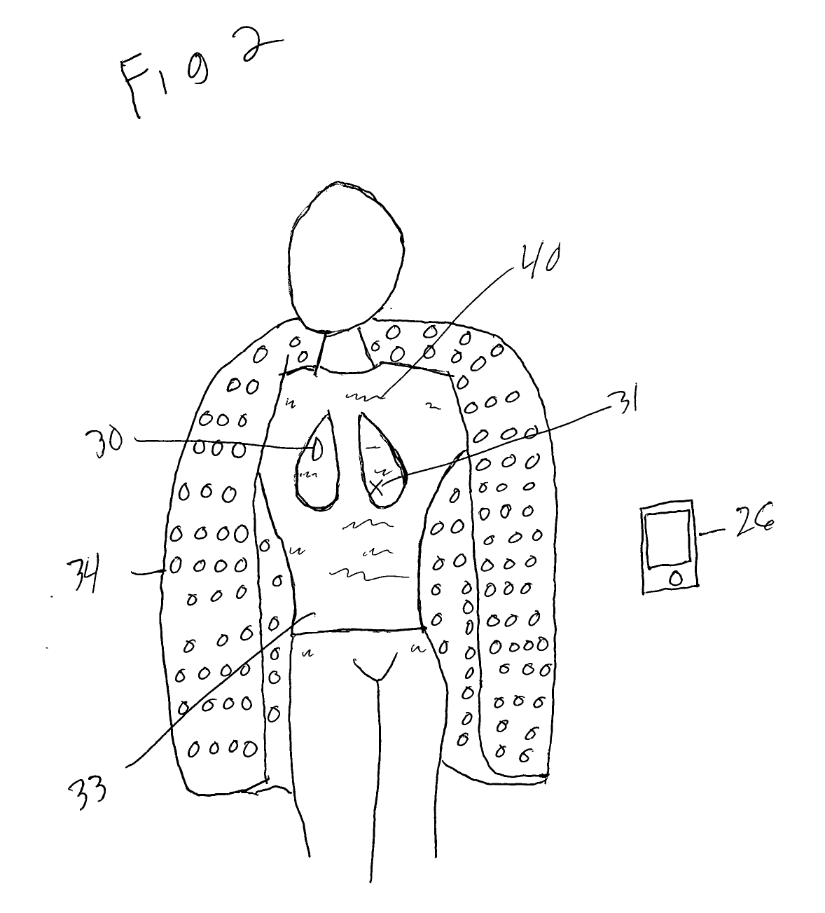

FIG.3 shows a handheld device scanning the body

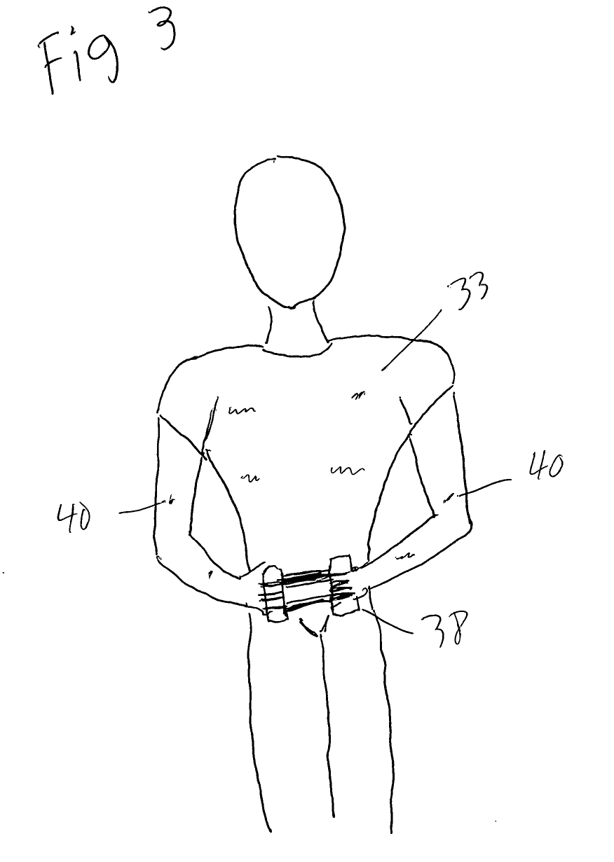

FIG. 4. shows a watch on both hands that scan and image the body

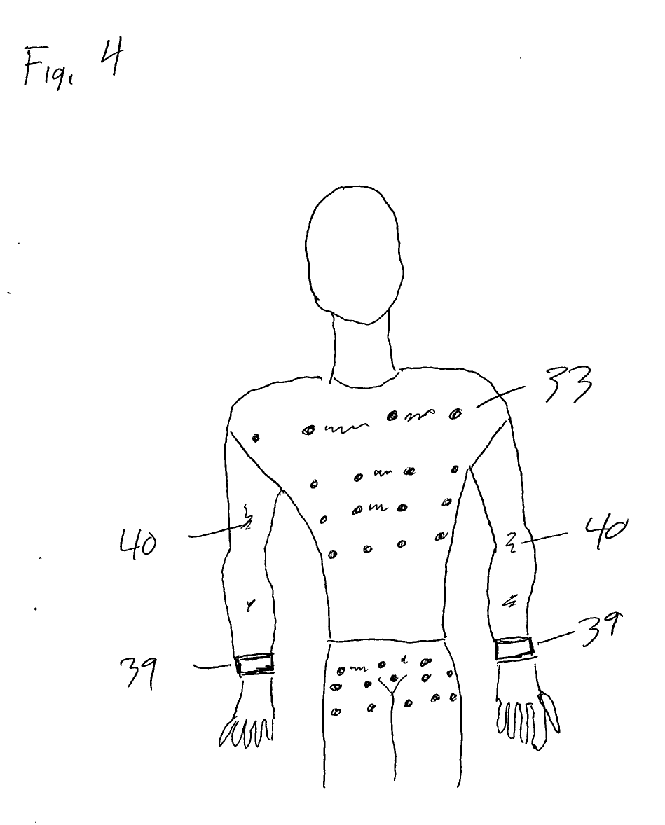

FIG. 5. shows a side view of a network of Electrical Impedance Tomography in a shirt

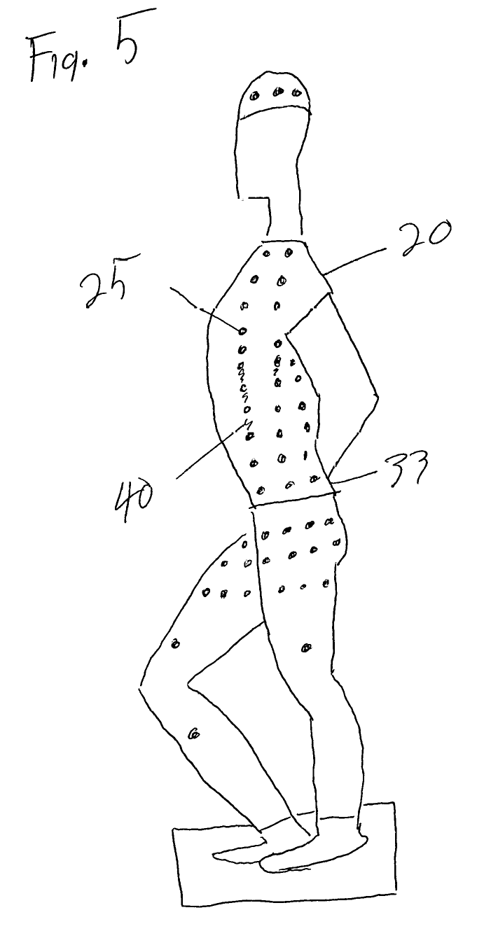

FIG. 6. shows a hat with a network of Electrical Impedance Tomography sensors

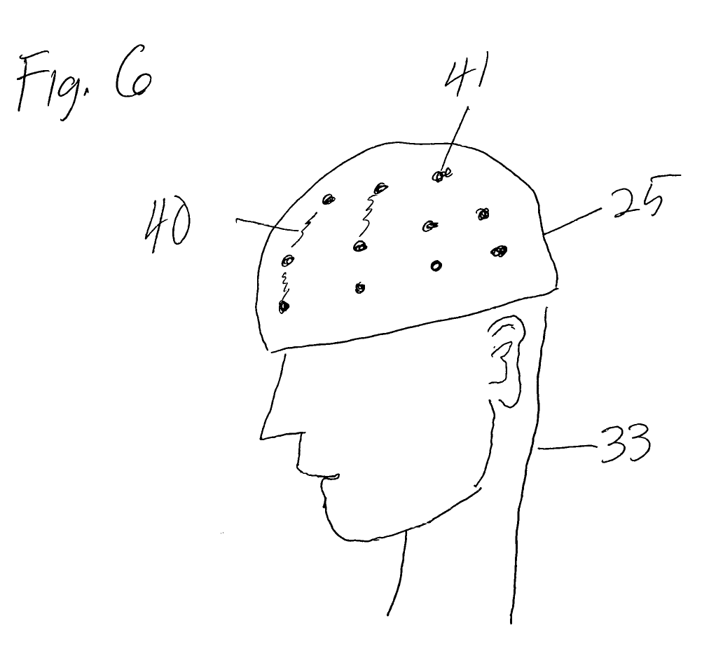

### DRAWING REFERENCE NUMBERS

FIG. 20 Shirt

FIG. 25 Sensor

FIG. 26 Smart Device

FIG. 28 cloud

FIG. 30 Cancer

FIG. 31 Pneumonia

FIG. 32 Lungs

FIG. 33 Human body

FIG. 34 Blanket

FIG. 36 wireless connection

FIG. 37 Internet connection

FIG. 38 handheld scanner

FIG. 39 Watch

FIG. 40 electrical current

FIG. 41 hat

We claim:

1. Method of detecting cancer using 2 or more Electrical Impedance sensors integrated into clothing connected to a smart device

2. Method of detecting pneumonia using a network of Electrical Impedance sensors integrated into wearable technology connected to a smart device

3. Method of imaging the inside of human body using a network Electrical Impedance sensors integrated into clothing connected to a smart device

4. Method of detecting cancer using software to analyze electrical impedance data from wearable technology

5. Method for scanning the human body using a network Electrical Impedance Tomography sensors integrated wearable technology

6. Means of imaging the internal human body using two or more electrical impedance sensors connected to a smart device

7. Means of detecting cancer using electrical impedance and device you stand on

8. Claim one can mean any device that you stand on to run the electrical impedance

9. Claim one can mean use a handheld device to control the electrical impedance to look for cancer tumors

10. claim 1. 2 and 3 can mean integrating sensors into clothing that connecting to the cloud through a smart device to image the inside of the human body

11. claims 1. 2 and 3 can clothing be a blanket

12. claims 1 and 2 can non-clothing devices can be used to detect cancer

13. claims 1 and 2 can uses handheld device for the scanning

14. claims 1. 2. and 3. can uses Watch or watches to do the scanning a living body

15. claims 1 and 2 uses app on smart device to scan and analyze the data

16. claims 1. 2. and 3 a human body can also mean animal body.

17. We claim a method for Imaging the internal human body using network of electrical impedance sensors integrated into wearable technology connected to a smart device and cloud computers.

18. We claim a new means of wearable technology that uses electrical impedance tomography to image internal human body

19. We claim a new means of Health monitoring using electrical impedance tomography to monitor an image a humans internal health
